(19) United States
(12) Patent Application Publication
(10) Pub. No.: US 2021/0275624 A1
(43) Pub. Date: Sep. 9, 2021

(54) TREATMENTS FOR IMPROVING OR LESSENING IMPAIRMENT OF MITOCHONDRIAL FUNCTION

(71) Applicant: Allegro Pharmaceuticals, LLC, San Juan Capistrano, CA (US)

(72) Inventors: Hampar L. Karageozian, San Juan Capistrano, CA (US); John Y Park, Santa Ana, CA (US); Vicken H. Karageozian, San Juan Capistrano, CA (US)

(21) Appl. No.: 17/193,832

(22) Filed: Mar. 5, 2021

Related U.S. Application Data
(60) Provisional application No. 62/986,361, filed on Mar. 6, 2020.

(51) Int. Cl. A61K 38/08; A61P 39/06

(52) U.S. Cl. CPC A61K 38/08; A61P 39/06

(57) ABSTRACT

Peptide therapies to improve, or lessen impairment of, mitochondrial function. These therapies are useable to disorders causing, caused by, contributing to, or related to mitochondrial dysfunction, such as neurodegeneration, metabolic disease, congestive heart failure, chronic heart failure with reduced ejection fraction, chronic heart failure with preserved ejection fraction, Barth syndrome, kidney disease and kidney failure due to percutaneous renal angiography for renal artery stenosis, impaired skeletal muscle function in the elderly, primary muscle mitochondrial myopathy and neuropathy, ischemia-reperfusion injury and protozoal infections, peripheral neuropathy, dermatologic disorders and inflamed hemorrhoids.

### RELATED APPLICATION

This patent application claims priority to U.S. Provisional Patent Application No. 62/986,361 entitled Peptide Treatments for Improving or Lessening Impairment of Mitochondrial Bioenergetics filed Mar. 6, 2020, the entire disclosure of which is expressly incorporated herein by reference.

### FIELD OF THE INVENTION

The present disclosure relates generally to the fields of chemistry, life sciences, pharmacy and medicine and more particularly to pharmaceutical preparations and their uses in the treatment disease.

### BACKGROUND OF THE INVENTION

Pursuant to 37 CFR 1.71(e), this patent document contains material which is subject to copyright protection and the owner of this patent document reserves all copyright rights whatsoever.

Risuteganib (sometimes referred to herein as RSG), is a non-natural peptide having the molecular formula C22-H39-N9-011-S and the following structural formula:

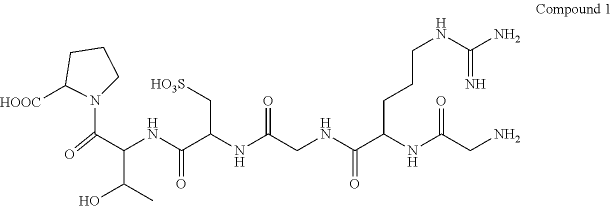 [SMILES: CC(O)C(NC(=O)C(CS(=O)(=O)O)NC(=O)CNC(=O)C(CCCNC(=N)N)NC(=O)CN)C(=O)N1CCCC1C(=O)O]

Risuteganib has also been referred to by other names, nomenclatures and designations, including: ALG-1001; Glycyl-L-arginylglycyl-3-sulfo-L-alanyl-L-threonyl-L-proline; Arg-Gly-NH—CH(CH₂—SO₃H)COOH; and Luminate® (Allegro Ophthalmics, LLC, San Juan Capistrano, Calif.). Risuteganib is an anti-integrin which has been shown to target a number of integrins upstream in the oxidative stress pathway. Risuteganib acts broadly to downregulate multiple angiogenic and inflammatory processes, including those associated with hypoxia and oxidative stress.

Additional description of and information relating to Risuteganib is provided in U.S. Pat. Nos. 9,018,352; 9,872,886; 9,896,480 and 10,307,460 and in United States Patent Application Publication Nos. 2018/0207227 and 2019/0062371 and co-pending U.S. Provisional Patent Applications No. 62/836,858 filed Apr. 22, 2019 entitled Compositions And Methods Useable For Treatment Of Dry Eye and 62/879,281 filed Jul. 26, 2019 entitled Peptides For Treating Dry Macular Degeneration And Other Disorders Of The Eye, the entire disclosure of each such patent and patent application being expressly incorporated herein by reference.

Mitochondria are organelles found within many types of cells. Mitochondria function to create energy by generating adenosine triphosphate (ATP), which fuels many of the body's functions. Because muscle and nerve cells have high energy demand, mitochondrial dysfunction is frequently manifested in the form of a muscular or neurological disorder. Mitochondrial disorders that primarily affect muscles are sometimes referred to as mitochondrial myopathies. Mitochondrial disorders that primarily affect nerves are sometimes referred to as mitochondrial neuropathies or mitochondrial encephalomyopathies. Mitochondrial dysfunction can contribute to a wide variety of disorders, such as neurodegeneration, metabolic disease, congestive heart failure, chronic heart failure with reduced ejection fraction, chronic heart failure with preserved ejection fraction, Barth syndrome, kidney disease and kidney failure due to percutaneous renal angiography for renal artery stenosis, skeletal muscle function in the elderly, primary muscle mitochondrial myopathy and neuropathy, ischemia-reperfusion injury and protozoal infections. Murphy, M.; Mitochondria as a Therapeutic Target for Common Pathologies; Nature Reviews: Drug Discovery, 2018 December; 17(12):865-886. doi: 10.1038/nrd.2018.174. Epub 2018 Nov. 5. Mitochondria provide both the energy and signals that enable and direct adaptation to stress on a cellular level. Thus, See, Picard, M., et al.: An Energetic View of Stress: Focus on Mitochondria; Frontiers in Neuroendocrinology 49 (2018) 72-85. Also, mitochondrial dysfunction has been identified as a factor in the development of autism spectrum disorders. See, Giulivi, et al., Mitochondrial Dysfunction in Autism. Journal of the American Medical Association. 2010; 304:2389-2396; Chauhan, et al., Brain region-specific deficit in mitochondrial electron transport chain complexes in children with autism. Journal of Neurochemistry 2011; 117:209-22; Tang, et al., Mitochondrial abnormalities in temporal lobe of autistic brain. Neurobiology of Disease 2013; 54:349-361; Goh, et al. Mitochondrial Dysfunction as a Neurobiological Subtype of Autism Spectrum Disorder: Evidence from brain imaging. JAMA Psychiatry 2014; 71:665-671; Napoli, et al., Deficits in bioenergetics and impaired immune response in granulocytes from children with autism. Pediatrics 2014; 133:e1405-10; Rose, et al., Oxidative stress induces mitochondrial dysfunction in a subset of autism lymphoblastoid cell lines in a well-matched case control cohort. PLOS One 2014; 9:e85436; Parikh, et al. A Modern Approach to the Treatment of Mitochondrial Disease. Current Treatment Options in Neurology. 2009; 11: 414-430; and Geier, et al. A prospective double-blind, randomized clinical trial of levocarnitine to treat autism spectrum disorders. Med Sci Monit 2011; 17:PI15-23.

There exists a need for the development of new therapies to improve, or lessen impairment of, mitochondrial function thereby treating diseases and disorders which are caused by or related to mitochondrial dysfunction.

### SUMMARY

The present disclosure describes treatments for improving mitochondrial function and/or for treating disorders that cause, are caused by, or which involve decreased or impaired mitochondrial function. The peptide may comprise Risuteganib or other peptides, examples of which are described herein.

In accordance with one aspect, there is provided a method for improving mitochondrial bioenergetics in a human or animal subject in need thereof, said method comprising the step of administering to the subject a therapeutically effective amount of a peptide which causes an improvement in mitochondrial bioenergetics. In some applications, the subject may be suffering from a disorder which causes, contributes to, or is caused by impairment of mitochondrial bioenergetics and the administration of the peptide reverses or prevents at least some of such impairment of mitochondrial bioenergetics such as, for example, a chemotoxic, hypoxic or ischemic insult, metabolic stress, heart failure, chronic heart failure with reduced ejection fraction, chronic heart failure with preserved ejection fraction, Barth syndrome, kidney disease, kidney failure due to percutaneous renal angiography for renal artery stenosis, impaired skeletal muscle function in the elderly, primary muscle mitochondrial myopathy or neuropathy, ischemia-reperfusion injury, protozoal infections, peripheral neuropathy, dermatologic disorders, neuronerative disease, Alzheimer's disease (AD), Parkinson's disease (PD), Huntington's disease (HD), amyotrophic lateral sclerosis (ALS), another disorder which causes progressive degeneration of function and/or structure of neurons of the central nervous system (CNS), a dermatologic disorder, a rash, pigmentation abnormality or acrocyanosis, pruritis, atopic dermatitis, psoriasis, a peripheral neuropathy, peripheral nerve pain, or nerve pain that causes, contributes to, or is caused by mitochondrial dysfunction, diabetes, abnormal glucose metabolism, oxidative stress or chemotherapy, heart failure or reduced cardiac output.

Still further aspects and details of the present disclosure will be understood upon reading of the detailed description and examples set forth herebelow.

### BRIEF DESCRIPTION OF THE DRAWINGS

The following figures are included in this patent application and referenced in the following Detailed Description. These figures are intended only to illustrate certain aspects or embodiments of the present disclosure and do not limit the scope of the present disclosure in any way:

FIG. 1 is a bar graph showing percentage of live vs. dead cells following treatment with control, RSG alone, HQ alone or RSQ+HQ co-treatment, indicating that RSG co-treatment significantly protected cells from HQ-induced reduction in cell viability.

FIG. 2A is a bar graph showing cell viability as reflected by mitochondrial dehydrogenase activity measured using the WST-1 reagent following treatment with control, RSG alone, HQ alone or RSQ+HQ co-treatment,

FIG. 2B is a bar graph showing mitochondrial respiratory parameters (i.e., basal respiration, maximal OCR, ATP production and spare respiratory capacity) following treatment with control, RSG alone, HQ alone or RSQ+HQ co-treatment.

FIG. 2C is a bar graph showing reactive oxygen species, as measured by relative fluorescence units (RFU) at the indicated times using a fluorescence plate reader following treatment with control, RSG alone, HQ alone or RSQ+HQ co-treatment.

FIG. 2D is a bar graph showing Δψm measured at the indicated times by the fluorescence of JC-1 monomers and aggregates in RPE cells following treatment with control, RSG alone, HQ alone or RSQ+HQ co-treatment, indicating that RSG co-treatment significantly improved the HQ-mediated reduction of Δψm.

FIG. 3 is a bar graph showing F-actin aggregates quantified with Fiji Image-J following treatment with control, RSG alone, HQ alone or RSQ+HQ co-treatment, indicating that HQ significantly induced RPE F-actin aggregation versus control, while RSG co-treatment significantly decreased HQ-induced aggregation.

FIG. 4 is a bar graph showing the number of expressed genes that passed FDR and log 2FC thresholds in RPE cells following treatment with control, RSG alone, HQ alone or RSQ+HQ co-treatment, indicating that few DE genes were found following RSG treatment alone, while treatment with HQ alone and the HQ+RSG co-treatment induced large transcriptome changes.

FIG. 5A is a bar graph showing HMOX-1 gene expression as measured by RNA-seq in RPE cells following treatment with control, RSG alone, HQ alone or RSQ+HQ co-treatment.

FIG. 5B is a bar graph showing HMOX-1 gene expression as measured by qPCR in RPE cells following treatment with control, RSG alone, HQ alone or RSQ+HQ co-treatment.

FIG. 5C is a Western Blot image showing quantity of HO-1 relative to GAPDH in RPE cells following treatment with control, RSG alone, HQ alone or RSQ+HQ co-treatment.

FIG. 5D is a bar graph showing the ratio of HQ-1 to GAPDH in RPE cells following treatment with control, RSG alone, HQ alone or RSQ+HQ co-treatment, indicating that the HQ-induced elevation in HO-1 protein level was further up-regulated by RSG co-treatment.

### DETAILED DESCRIPTION OF THE INVENTION

The following detailed description and the accompanying drawings to which it refers are intended to describe some, but not necessarily all, examples or embodiments of the invention. The described embodiments are to be considered in all respects only as illustrative and not restrictive. The contents of this detailed description and the accompanying drawings do not limit the scope of the invention in any way.

As used herein the term “patient or “subject” refers to human or non-human animals, such as humans, primates, mammals, and vertebrates.

As used herein the term “treat” or “treating” refers to preventing, eliminating, curing, deterring, reducing the severity or reducing at least one symptom of a condition, disease or disorder.

As used herein the phrase “effective amount” or “amount effective to” refers to an amount of an agent that produces some desired effect at a reasonable benefit/risk ratio. In certain embodiments, the term refers to that amount necessary or sufficient to treat a specified condition or disorder. The effective amount may vary depending on such factors as the disease or condition being treated, the particular composition being administered, or the severity of the disease or condition. Persons of skill in the art may empirically determine the effective amount of a particular agent without necessitating undue experimentation.

The treatments described in this patent application may be administered by any suitable route(s) of administration and in any suitable dosage form. Possible routes of administration known in the art include, but are not necessarily limited to: systemic, local, regional, parenteral, enteral, inhalational, topical, intramuscular, subcutaneous, intravenous, intra-arterial, intrathecal, intravesical, oral, endoscopic (e.g., through an endoscope, bronchoscope, colonoscope, sigmoidoscope, hysterscope, laproscope, athroscope, gastroscope, cystoscope, etc.), transurethral, rectal, nasal, oral, tracheal, bronchial, esophageal, gastric, intestinal, peritoneal, urethral, vesicular, urethral, vaginal, uterine, fallopian, buccal, lingual, sublingual and mucosal. Possible dosage forms known in the art include, but are not limited to: liquids, biphasic liquids, solids, semisolids, vapors, aerosols, solutions, suspensions, mixtures, syrups, linctuses, gels, creams, pastes, ointments, lotions, liniments, collodions, emulsions, transdermal delivery patches, suppositories, capsules, tablets, powders, granules, edibles, chewables, drops, sprays, enemas, douches, lozenges, etc.

### Example 1

### Risuteganib (RSG) Protects Against Hydroquinone-Induced Injury in Human Retinal Pigment Epithelium RPE Cells

Cigarette smoking has been implicated in the pathogenesis of age-related macular degeneration (AMD). Integrin dysfunctions have been associated with AMD. Applicants have investigated the effect of Risuteganib on RPE cell injury induced by hydroquinone (HQ), an important oxidant in cigarette smoke.

Cultured human RPE cells were treated with HQ in the presence or absence of RSG. Cell death was evaluated by flow cytometry. Mitochondrial respiration was measured by XFe24 analyzer. Reactive oxygen species (ROS) production and mitochondrial membrane potential (Δψm) were quantified by fluorescence plate reader. F-actin aggregation was visualized with phalloidin. Whole transcriptome analysis and gene expression were analyzed by Illumina RNA sequencing and qPCR, respectively. Levels of heme oxygenase-1 (HO-1) protein were measured by Western blot.

HQ induced necrosis and apoptosis, decreased mitochondrial bioenergetics, increased ROS levels, decreased Δψm, and increased F-actin aggregates. RSG co-treatment significantly protected against HQ-induced necrosis and apoptosis, prevented HQ-reduced mitochondrial bioenergetics, decreased HQ-induced ROS production, improved HQ-disrupted Δψm, and decreased HQ-induced F-actin aggregates. RSG alone minimally affected the RPE cell transcriptome. HQ induced substantial transcriptome changes. A variety of biological processes induced by HQ were regulated by RSG co-treatment. HO-1 protein levels were significantly up-regulated by HQ and further up-regulated by RSG co-treatment.

In this study, RSG had no detectable adverse effects on healthy RPE cells, while RSG co-treatment protected against HQ-induced injury and mitochondrial dysfunction. RSG co-treatment modified a variety of the biological processes induced by HQ, suggesting a potential role for RSG therapy to treat retinal diseases such as AMD.

RPE cells form a monolayer of highly specialized, polarized epithelial cells interposed between the choriocapillaris and photoreceptors. RPE cells play an important role in retinal homeostasis and are vital to photoreceptor cell health and visual function.

RPE cell dysfunction or death is thought to be an important contributor to age-related macular degeneration (AMD). RPE cells are continually exposed to oxidants throughout life and oxidative stress plays a major role in AMD pathogenesis and progression. Cigarette smoke contains high concentrations of free oxidants and has been implicated as a major environmental risk factor for AMD. Hydroquinone (HQ), a major oxidant in both tobacco smoke and atmospheric pollutants, increases reactive oxygen species (ROS) generation and promotes oxidative stress.: ROS, a group of unstable oxygen-containing molecules that can easily react with other molecules in a cell, are generated during cellular metabolism and in response to various stimuli. In cells, the major site of ROS production is the mitochondrial electron transport chain, where some electrons leak from the transport process and spontaneously react with molecular oxygen, producing superoxide anion. ROS have important physiological functions; however, excess ROS can cause RPE cell oxidative damage.

Mitochondria, the intracellular organelles comprising the main respiratory machinery in cells, are crucial for energy production and cell homeostasis. Due to a high level of metabolic demand by photoreceptors, RPE cells are enriched with a large mitochondrial population to meet the high-energy needs. Consequently, RPE mitochondrial dysfunction can lead to tissue damage and has been implicated in the development of AMD. In RPE cells from eyes with AMD, damaged, fragmented, and ruptured mitochondria have been observed. mtDNA mutation levels are also elevated in RPE cells of eyes with AMD.

Integrins, a family of heterodimeric, non-covalently bound cell adhesion proteins, are transmembrane receptors consisting of a larger a and a smaller β subunits. Integrins serve as bridges between cells and regulate cellular interaction with other surrounding cells and with the extracellular matrix through signal transduction pathways. Physiologically, they play important roles in cell adhesion, proliferation, shape and motility. Integrins αvβ3, αvβ5, and α5β1 are closely associated with choroidal neovascularization in eyes with wet AMD and with pre-retinal neovascularization in eyes with diabetic retinopathy and macular edema. Integrins serve as adhesion molecules, mechanosensors, and signal transduction platforms in a variety of biological processes, and are also central to the etiology and pathology of many disease states. Therefore, integrins are an attractive target to treat retinal diseases.

Risuteganib is an engineered arginylglycylaspartic acid (RGD) class synthetic peptide that modulates integrin receptors. RGD peptide treatment suppresses retinal neovascularization and facilitates the release of cellular adhesion between the vitreous and the retina, inducing posterior vitreous detachment. In previous studies, RSG reduces expression of several disease-relevant integrins including αvβ3, αvβ5, α2β1, and α5β1, which are expressed in RPE cells. Earlier studies also suggest a potential cytoprotective effect of RSG, which would be beneficial in retinal degenerative diseases such as dry AMD. Herein, we investigated the effect of RSG on HQ-induced RPE cell injury, in an in vitro cell culture system.

RSG was obtained from Allegro Ophthalmics, LLC (San Juan Capistrano, Calif., USA). HQ, the tetrazolium salt WST-1 (4-[3-(4-Iodophenyl)-2-(4-nitrophenyl)-2H-5-tetrazolio]-1.3-benzene disulfonate), phalloidin-TRITC (cat #P1951), and collagen type I were purchased from Sigma (St. Louis, Mo., USA). TrypLE (cat #12604-013), Annexin V Pacific Blue™ conjugate (cat #A35122), Annexin V binding buffer (cat #V13246), eBioscience™ 7-AAD viability staining solution (cat #00-6993-50), JC-1 dye and 5-(and-6)-chloromethyl-2′,7′-dichlorodihydrofluorescein diacetate, acetyl ester (CM-H2DCFDA), BCA protein assay kit, radioimmunoprecipitation assay (RIPA) lysis and extraction buffer (cat #89900), and TURBO DNA-free kit were purchased from Thermo Scientific (Chicago, Ill., USA). Cell mito stress test kit (cat #103015-100), XF base medium (cat #103193-100), and XFe24 FluxPak (cat #102342-100) were purchased from Agilent technologies (Santa Clara, Calif., USA). RNeasy Mini and RNeasy Plus Mini Kits were purchased from Qiagen (Valencia, Calif., USA). Rabbit anti-heme oxygenase-1 (HO-1, cat #ADI-OSA-150D) antibody was purchased from Enzo Life Sciences (Farmingdale, N.Y., USA). Mouse anti-GAPDH antibody was purchased from Chemicon (Temecula, Calif., USA).

### Human RPE Cell Culture and Treatments

Human donor eyes from a 62-year-old male donor were obtained within 24 hrs. after death from the North Carolina Organ Donor and Eye Bank, Inc. (Winston-Salem, N.C., USA) in accordance with the provisions of the Declaration of Helsinki for research involving human tissue. Cells were grown in Eagle's minimal essential medium (MEM; Invitrogen) with 10% fetal bovine serum (FBS; Thermo Scientific) and with 1× penicillin/streptomycin (Thermo Scientific) at 37° C. in a humidified environment containing 5% CO₂. The identity of RPE cells was confirmed by cytokeratin-18 and ZO-1 stain (not shown).

Human donor RPE cells were seeded on collagen-coated 96-well plates, 24-well plates with or without coverslips, 6-well plates (Corning-Costar Incorporated, Corning, N.Y., USA). Except for the Seahorse assay, on day 6 after plating, cells were washed twice with serum-free and phenol red-free MEM (SF-MEM), treated with HQ at a various concentrations in the presence or absence of RSG (0.4 mM) in SF-MEM for various times at 37° C. RSG co-treatment refers to the condition in which cells were treated with RSG in the presence of HQ. Control refers to the condition in which cells were not treated.

### Flow Cytometry

RPE cells in triplicate wells of a 6-well plate were treated with HQ (150 μM) in the presence or absence of RSG (0.4 mM) for 12 hours. Cell morphology was recorded by light microscopy, and then cells were detached with 1×TrypLE and centrifuged at 300 g for 5 minutes. Cells were re-suspended with 100 μL 1×Annexin V binding buffer, incubated with 5 μL Annexin V for 10 minutes and then 5 μL 7-AAD was added to the Annexin V mixture and incubated for additional 5 minutes. Cell death was analyzed with flow cytometry.

### WST Assay

RPE cells in triplicate wells of a 96-well plate were treated with HQ (150 μM) for 2.5 hours in the presence or absence of RSG (0.4 mM). The medium was removed and cells were incubated with WST-1 solution for 30 minutes at 37° C. A colorimetric assay was performed based on the cleavage of the tetrazolium salt WST-1 by mitochondrial dehydrogenases in viable cells. The plate was read on a spectrophotometer at 440 nm with a reference wavelength at 690 nm.

### Seahorse Assay

RPE cells were seeded in triplicate wells of collagen-coated XF 24-well plates and grown for 24 hours. RPE cells that had just reached confluence were washed with SF-MEM and treated for 1.5 hours with HQ (175 μM) with or without RSG (0.4 mM). Media were removed and cells were washed with XF base medium containing 1 mM sodium pyruvate, 2 mM glutamine and 8 mM glucose, pH 7.4. The cells were incubated for 1 hour at 37° C. in a C02-free incubator. Oxygen consumption rate (OCR) was measured by Seahorse XFe24 flux analyzer under basal conditions followed by the sequential addition of 1 μM oligomycin, 1 μM tri-fluorocarbonylcyanide phenylhydrazone (FCCP), and 1 μM rotenone and antimycin A. Maximal OCR was the difference in OCR between FCCP-induced respiration and OCR after injection of antimycin A. Mitochondrial spare respiratory capacity was the difference between maximal respiration and basal OCR. Media were removed and the total proteins were extracted for BCA protein assay after OCR measurements. OCRs were normalized to total protein content.

### Determination of ROS

RPE cells in triplicate wells of a 96-well black plates with clear bottoms were washed with SF-MEM, loaded with 20 μM CM-H2DCFDA in SF-MEM for 30 minutes at 37° C. and then washed twice. Cells were then treated with HQ (160 μM) in the presence or absence of RSG (0.4 mM). Fluorescence was measured at various times with a fluorescence plate reader (490 nm excitation, 522 nm emission).

### Determination of Δψm

RPE cells in triplicate wells of 96-well black plates with clear bottoms were washed with SF-MEM, loaded with 10 μM JC-1 dye in SF-MEM for 30 minutes at 37° C. and then washed twice. Cells were then treated with HQ (160 μM) with or without RSG (0.4 mM). A fluorescence plate reader was used to measure the fluorescence at various times to quantify green JC-1 monomer (490-nm excitation, 522-nm emission) and red JC-1 aggregates (535-nm excitation, 590-nm emission).

### F-Actin Immunofluorescent Staining

RPE cells in triplicate wells of a 24-well plate containing collagen-coated coverslips were treated for 6 hours with HQ (140 μM) in the presence or absence of RSG (0.4 mM) and then fixed in 4% paraformaldehyde for 12 minutes at room temperature (RT). Cells were permeabilized with 0.5% Triton X-100 for 12 minutes and incubated for 1 hour at RT with phalloidin-TRITC in 0.5% bovine serum albumin/0.1% Triton X-100/PBS. The percentage of F-actin aggregates was determined by a masked observer. Briefly, for each sample, ten to eleven Z-stack images (covering 12 μm at 1 μm z-steps, 6 along the x-axis and 5 along the y-axis non-overlapping fields) were captured under identical conditions with a 20× objective on a Nikon A1 confocal microscope. Maximum intensity projected images were imported into Fiji software and segmented using the Trainable Weka Segmentation plugin following Fiji online instructions. Briefly, for generation of a “threshold”, a segmentation classifier was created using an image containing both aggregates and non-aggregates. Aggregates and non-aggregates were traced and added into separate groups, respectively. This process was repeated until satisfactory image segmentation was achieved. The classifier was then tested on three images that had varying degrees of aggregate density to ensure accurate segmentation. For data analysis, segmented images were converted to eight-bit, binary images and F-actin aggregates were quantified using the particle analyzer function (0-infinity size and 0-1 circularity) in Fiji.

### RNA-Sequencing (RNA-Seq) Sample Preparation and Analysis

RPE cells in sextuplicate wells of a 6-well plate were treated for 4 hours with HQ (250 μM or 300 μM) in the presence or absence of RSG (0.4 mM). Total RNA was extracted using an RNeasy Mini Kit and DNA was removed with TURBO DNA-free kit. RNA quality was measured with a Bioanalyzer (Agilent Genomics, Santa Clara, Calif., USA). RNA-seq libraries were prepared from poly-A enriched messenger RNA and sequenced by GENEWIZ (South plainfield, NJ, USA) to generate approximately 20-30 million paired-end, 150 base-pair reads per sample. RNA-seq FASTQ files were quality-tested with FastQC. Reads were aligned with STAR to human genome (GRCh38.p12) and transcriptome (GENECODE v30) references, followed by read-quantification with featureCounts.

Principal component analysis (PCA) was used to visualize the high-dimensional RNA-seq dataset using the top 15 thousand genes that had the highest average counts, as normalized by DESeq2's VST method. Differential expression analysis performed using edgeR exact test, was limited to genes with count per million (CPM)≥1 in at least 6 samples in the dataset. Differentially expressed (DE) genes are defined to have false discovery rate (FDR)<0.05 and absolute value of log 2-fold-change (log 2FC)>0.25. Strongly regulated DE genes, defined as those with absolute value of log 2FC>0.50, were submitted to goseq for enrichment of Gene Ontology (GO) biological processes and Kyoto Encyclopedia of Genes and Genomes (KEGG) biological pathways that are over-represented in the gene list. Biological processes and pathways were considered statistically significant with adjusted p-value <0.05. Enriched biological processes were condensed and visualized with REVIGO with similarity metric set to small and GO term size determined by Uniport Homo Sapiens database; biologically relevant processes were selected and labeled. Six enriched KEGG pathways were selected for visualization of gene expression changes, based on their biological relevance and strength of negative correlation between HQ and RSG co-treatment. Empirical RNA-seq Sample Size Analysis (ERSSA) was used to check whether the six biological replicates used in this study were sufficient for differential expression discovery; analysis was performed with log 2FC cutoff of 0.25 and 50 subsamples at each replicate level.

### Real-Time RT-PCR Analysis

RPE cells in triplicate wells of a 12-well plate were treated with HQ (250 μM) in the presence or absence of RSG (0.4 mM) for 4 hours. Total RNA was isolated using RNeasy Plus Mini Kit according to the manufacturer's specifications and real-time quantitative reverse transcription-polymerase chain reaction (qPCR) was performed as we have described previously. Primer pairs for HMOX-1 and ribosomal protein, large, PO (RPLPO) were as follows (5′ to 3′): HMOX-1, forward: CAG GAG CTG ACC CAT GA; reverse: AGC AAC TGT CGC CAC CAG AA; RPLPO, forward: GGA CAT GTT GCT GGC CAA TAA; reverse: GGG CCC GAG ACC AGT GTT.

### Western Blot

RPE cells in triplicate wells of a 6-well plate were treated with HQ (170 μM) in the presence or absence of RSG (0.4 mM) for 8 hours. Total protein were extracted with RIPA buffer supplemented with protease inhibitors and phosphatase inhibitors, quantified with Bradford protein assay, and subjected to SDS-PAGE and Western blot as we have previously described.²¹

### Statistical Analysis

Data are expressed as the mean±SD. Two-way ANOVA with Tukey multiple comparisons correction was used to determine whether there were statistically significant differences between treatment groups as measured by flow cytometry, WST assay, XFe24 flux analyzer, ROS assay, JC-1 assay, F-actin aggregates analysis, qPCR, and Western blot. Results were plotted using GraphPad Prism 8.3.0 with asterisks indicating the magnitude of p-value (N.S.=not significant, *P≤0.05, **P≤0.01, ***P≤0.001, ****P≤0.0001).

### Results

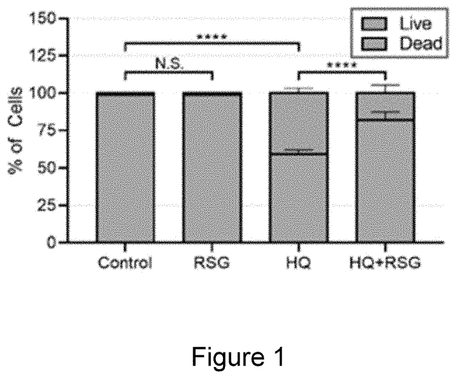

In this study, RSG co-treatment protected against HQ-mediated RPE cell death. To investigate whether RSG protected against HQ-induced RPE cell death, flow cytometry analysis was performed with annexin V and 7-AAD staining to identify apoptosis and necrosis, respectively. Early apoptotic cells are Annexin V positive and 7-AAD negative. Late apoptotic cells are Annexin V and 7-AAD double positive. Necrotic cells are 7-AAD positive and Annexin V negative. As shown in FIG. 1A, HQ (150 μM) caused cells to shrivel/detach, while RSG co-treatment improved HQ-induced morphological changes. Similar morphology was observed in cells treated with RSG alone, when compared to cells without treatment (control). FIG. 1 is a bar graph which shows percentage of live vs. dead cells following each experimental treatment (i.e., control, RSG alone, HQ alone, RSQ+HQ co-treatment). HQ alone induced primarily necrotic cell death and much less apoptotic cell death. RSG co-treatment significantly protected cells from HQ-induced necrosis and apoptosis. RSG alone had little effect on number of live and dead cells when compared to control.

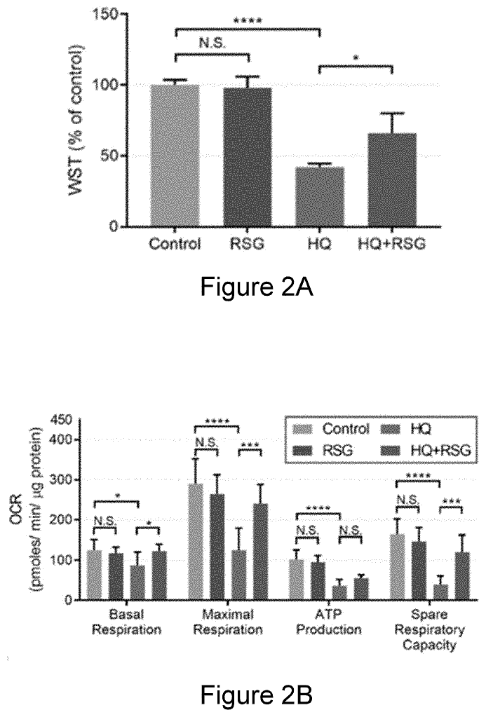

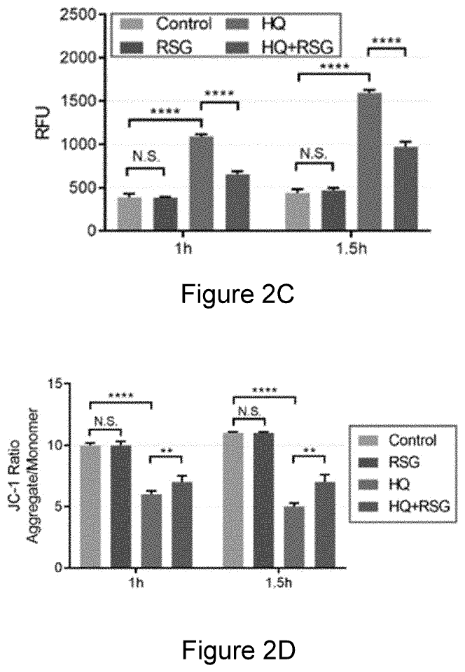

RSG co-treatment also protected cells from deleterious effects of HQ on mitochondria. WST-1 is a stable tetrazolium salt that can be cleaved by cellular mitochondrial dehydrogenase to form a soluble formazan. The amount of formazan generated by mitochondrial dehydrogenase activity is directly proportional to the number of living cells, which makes it useful as a cell viability assay. Accordingly, to further evaluate RSG's cytoprotective effect, a WST assay was performed. FIG. 2A is a bar graph showing the WST content of cells following each experimental treatment (i.e., control, RSG alone, HQ alone, RSQ+HQ co-treatment). As shown in FIG. 2A, HQ significantly decreased cell viability as reflected by decreased mitochondrial dehydrogenase activity, while RSG co-treatment significantly increased cell viability when compared to HQ alone. RSG alone had little effect on cell viability when compared to control.

Mitochondrial respiration plays an important role in cell survival. We evaluated the role of RSG in the regulation of mitochondrial function. As shown in FIG. 2B, HQ significantly decreased mitochondrial bioenergetics when compared to control. No significant change in mitochondrial bioenergetics was detected in cells treated with RSG alone, when compared to control. RSG co-treatment significantly increased basal respiration, maximal respiration, ATP production and spare respiratory capacity when compared to HQ-treated cells.

Mitochondria are major sources of ROS generation that contribute to oxidative stress-mediated cell death. To examine whether RSG reduces oxidative stress-mediated ROS production, we measured ROS levels. As shown in FIG. 2C, ROS production was significantly increased in HQ-treated cells when compared to control, while RSG co-treatment significantly decreased HQ-induced ROS production. RSG alone did not significantly change ROS level when compared to control.

Δψm is critical to maintain the physiological function of the respiratory chain to generate ATP. The membrane-permeant JC-1 dye is widely used to test Δψm. To evaluate the effect of RSG on HQ-mediated changes in Δψm, we determined the ratio of JC-1 monomers to aggregates. As shown in FIG. 2D, Δψm was significantly decreased in HQ-treated cells when compared to control, while RSG co-treatment significantly improved HQ-mediated reduction in Δψm. When compared to control, RSG alone did not induce significant changes in Δψm.

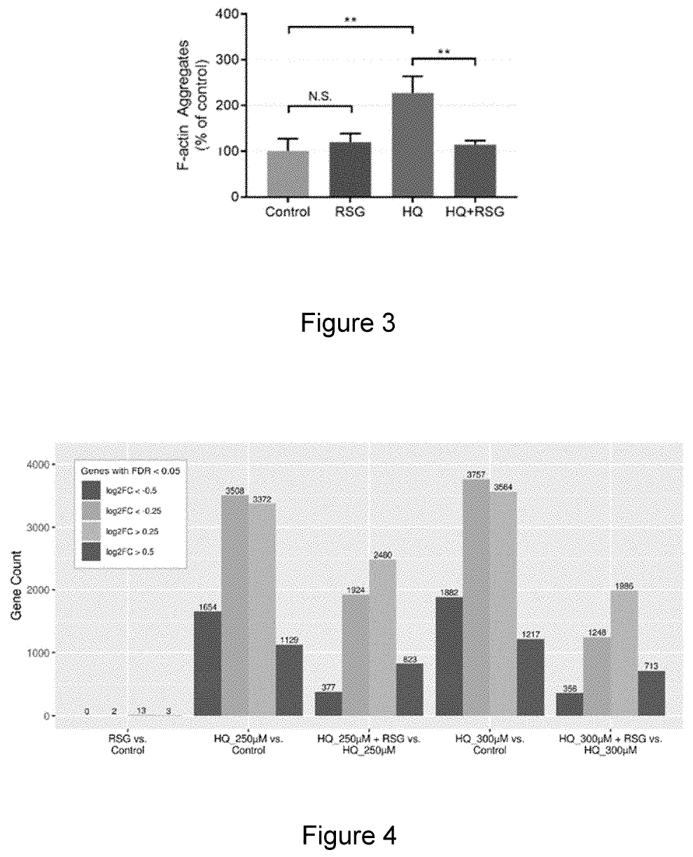

In this study, RSG co-treatment also decreased HQ-induced F-Actin aggregation. Regulation of F-actin plays important role in cell death. To determine whether RSG affected F-actin cytoskeleton organization, cells were treated with HQ with or without RSG followed by quantification of F-actin aggregates. As seen in the graph of FIG. 3, HQ significantly increased the number of F-actin aggregates when compared to control, while RSG co-treatment significantly inhibited the production of HQ-induced aggregates. RSG alone had no observable effect on F-actin cytoskeleton morphology and no significant change in number of F-actin aggregates when compared to control.

RSG co-treatment also modulated the HQ-regulated transcriptome. To investigate gene expression changes in response to HQ and RSG, RPE cells were exposed for 4 hours to HQ at two dosages (250 μM or 300 μM) with or without RSG, followed by whole transcriptome measurement using RNA-seq. All RNA-seq samples had excellent RNA quality (Bioanalyzer RNA Integrity Number >9.70) and sequencing quality. PCA visualization of the samples showed that the top 2 dimensions captured a combined 81% of variance in the dataset and revealed clear separation on dimension 1 between HQ-treated and non-treated cells and on dimension 2 between HQ-treated and RSG co-treated cells. No observable separation was discovered between cells treated with RSG alone and control.

As shown graphically in FIG. 4, on differential expression analysis, approximately seven thousand genes were changed in HQ-treated cells at the two dosages tested when compared to control, while RSG treatment alone only changed 15 DE genes. However, compared to cells treated with HQ alone, RSG co-treatment also regulated thousands of genes. Further analysis showed that many of the genes influenced by RSG co-treatment were also regulated by HQ and the expression frequently changed in the opposite direction to that induced by HQ. Additionally, when all HQ-regulated genes were evaluated, negative correlations were observed between expression changes modulated by HQ and by RSG co-treatment, indicating that RSG modulates HQ-induced expression changes.

Functional analysis of the DE gene lists was performed to identify biological processes and pathways that are significantly enriched in genes regulated by HQ, RSG treatment alone, or RSG co-treatment. Among the 15 DE genes found regulated by RSG treatment alone, no processes or pathways were enriched. When performed with genes regulated by either HQ or RSG co-treatment, a large number of processes were enriched including cell proliferation, cell death, regulation of cell adhesion, regulation of metabolism, inflammatory response and response to stimulus. In particular, a large number of processes overlapped between the two analyses, indicating that many of the same biological processes are regulated by HQ and RSG co-treatment. Similarly, enriched biological pathways showed large overlap between HQ and RSG co-treatment. Although many of the same biologically relevant pathways were regulated by HQ and RSG co-treatment, their induced changes were generally in opposite directions as indicated by the negative correlations. Overall, gene expression changes across the two dosages of HQ tested by RNA-seq were highly consistent as shown by the large positive correlations.

For the five differential expression comparisons tested, RNA-seq sample size analysis was performed using ERSSA. Briefly, ERSSA determines if sufficient biological replicates have been employed to discover a majority of DE genes in any specific comparison. ERSSA showed that sufficient samples were used in four of the comparisons to produce meaningful numbers of DE genes, as the average discovery trends tapered off when sample size approached n=6, the number of biological replicates per condition in this experiment. In the comparison of RSG vs. control, ERSSA showed that additional replicates were unlikely to further improve discovery beyond low double-digit number of DE genes.

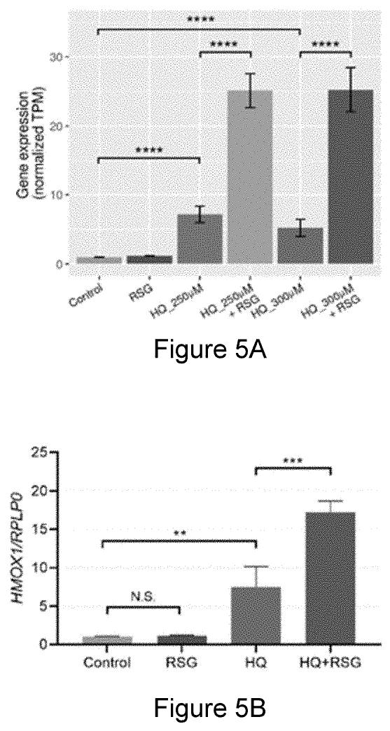

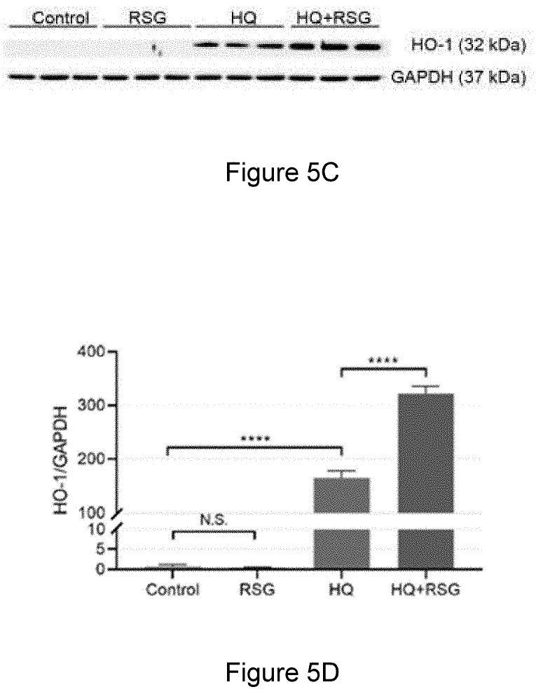

As shown in FIGS. 5A through 5D, RSG co-treatment also up-regulated HQ-induced HQ-1 expression. From RNA-seq data, there was significant up-regulation of the cytoprotective HMOX-1 gene that encodes HO-1 protein after HQ exposure (618% and 420% increase by 250 μM and 300 μM HQ, respectively), which was further enhanced by RSG co-treatment (241% and 391% increase) when compared to 250 μM and 300 μM HQ alone-induced expression levels.

To confirm RNA-seq results, HMOX-1 gene and HO-1 protein expressions were examined by qPCR and Western blot, respectively. HMOX-1 gene and HO-1 protein levels were significantly up-regulated by HQ and further up-regulated by RSG co-treatment.

Based on the above-summarized results, in this study, RSG treatment alone had no detectable adverse effect on healthy cells while RSG co-treatment significantly: 1). protected against HQ-induced RPE cell necrosis and apoptosis; 2). improved HQ-induced cell viability reduction; 3). prevented HQ-decreased mitochondrial bioenergetics; 4). suppressed HQ-induced ROS production; 5). improved HQ-disrupted Δψm; 6). mitigated HQ-induced F-actin aggregates. Furthermore, RSG alone minimally affected the RPE cell transcriptome, while HQ induced substantial transcriptome changes. RSG co-treatment modified HQ-regulated biological processes and pathways including calcium signaling, TGF-β signaling, Ras signaling, cytokine and receptor interaction, focal adhesion, and actin cytoskeleton. HMOX-1 mRNA and HO-1 protein levels were significantly up-regulated by HQ and further up-regulated by RSG co-treatment.

In age related macular degeneration, oxidative stress is believed to play a significant role in causing RPE cell dysfunction and death. The mechanism by which RPE oxidative stress induces death in this disease is still controversial. Most in vitro studies indicate that oxidative stress induces primarily apoptotic cell death, while some suggest that necrosis contributes to RPE death. Caspase 3/7 activity, measured as an apoptosis marker, is not changed in ARPE-19 cells after treatment with various HQ concentrations (50-500 μM) (Woo G B, et al. IOVS 2012; 53:ARVO E-abstract 4276). In contrast, HQ down-regulates apoptosis-related genes after HQ injury in differentiated ARPE-19 cells. In the current study, it was determined that HQ primarily induced necrosis and to a much lesser extent, apoptosis. RSG co-treatment significantly protected against both necrosis and apoptosis induced by HQ. Integrins trigger a variety of cell signaling cascades which have profound effects on cell viability. Integrins also play a complex role to regulate cell survival and their dysfunction can lead to apoptosis. These results suggest that RSG plays a role to protect against oxidative stress-induced integrin-mediated RPE necrosis and apoptosis.

Apoptosis is a highly regulated process. However, a significant component of necrotic death also occurs through highly regulated mechanisms. Necrosis can include signs of controlled processes such as mitochondrial dysfunction, enhanced generation of ROS, ATP depletion, and plasma membrane failure. Thus, mitochondrial dysfunction plays an important role in both apoptosis and necrosis. HQ increases ROS levels and decreases Δψm in ARPE-19 cells. The results obtained from donor RPE in the current study were consistent with data from ARPE-19 cells. In Seahorse assays, we found that HQ significantly decreased mitochondrial basal respiration, maximal respiration, ATP production, and spare respiratory capacity. RSG co-treatment significantly protected cells from deleterious effects of HQ on mitochondrial bioenergetics. These observations of mitochondrial protection are consistent with studies of RSG in human Muller cells and ARPE-19 cells. (Kenney C M, et al. IOVS 2018; 59:ARVO E-Abstract 771) (Beltran M A, et al. IOVS 2018; 59:ARVO E-Abstract 1465) Due to the important role mitochondria play in cellular function, integrin signaling can have a profound impact on the mitochondria and vice versa. Integrins control mitochondrial function and ROS production by interacting with small G proteins Integrin ligations trigger ROS production by promoting changes in mitochondrial metabolic/redox function and activation of distinct oxidases. Conversely, disruption of mitochondrial function with inhibitors increases ROS production and up-regulates integrin P5 expression in human gastric cancer SC-M1 cells. Our results suggest that RSG, as an integrin regulator, can positively influence mitochondrial function, although integrin-independent effects cannot be ruled out.

Actin cytoskeleton serves mechanical, organizational and signaling roles that contributes to cellular functions. The regulation of F-actin cytoskeleton depends on a variety of molecular mechanisms. Oxidative stress is a key mechanism that causes aggregation of a number of proteins implicated in neurodegenerative disorders. HQ induces F-actin aggregation through the phosphorylation of p38 mitogen-activated protein kinase (MAPK)/heat shock protein 27 (Hsp27) cascade in ARPE-19 cells. In the current study, we found HQ also induced F-actin aggregation in donor RPE cells and RSG co-treatment decreased HQ-induced aggregation. Actin cytoskeletal signaling networks are composed of a large group of proteins such as integrins, kinases, and small GTPases. Integrins can affect the actin cytoskeleton through a number of molecular linkages Integrins can either stimulate or suppress actin-based structures, indicating the variety of pathways leading from integrins to the cytoskeleton. We hypothesize that RSG prevents HQ-induced damage to normal actin cytoskeleton, at least in part by reversing adverse effects of HQ on integrin regulated F-actin assembly. Although it is beyond the scope of this paper, experiments are currently underway in our laboratory to test this hypothesis.

RNA-seq is a powerful quantitative tool to explore genome-wide expression. To further investigate RSG's therapeutic mechanism of action, we employed RNA-seq as an unbiased method to assess the transcriptome changes induced by the HQ and RSG co-treatment. We found RSG alone had minimal influence on the transcriptome, consistent with its limited effect on healthy RPE cells. A large transcriptome change involving roughly half of the detectable expressed genes was seen after HQ treatment. These observations are supported by our pathway analysis that showed regulation of genes in: 1). inflammation pathways that included cytokine-cytokine receptor interaction, JAK-STAT and TNF signaling pathways; 2). cell proliferation and death pathways such as apoptosis, PI3K-Akt, MAPK, TGF-beta and Ras signaling pathways; 3). cell adhesion and migration pathways such as focal adhesion and actin cytoskeleton regulation; 4) pathways that function in diverse signaling systems such as the calcium signaling pathway. These findings are consistent with studies in various cell models that show regulation of cell growth, cell death, extracellular matrix and stress response genes after HQ exposure.

While RSG alone elicited minimal transcriptome changes, RSG co-treatment significantly modified the cellular gene expression response to HQ stress. A significant portion of the RSG-regulated genes under HQ stress were also regulated by HQ treatment alone, except the expression changes were in the opposite direction. Similarly, functional analysis of RSG co-treatment-regulated genes indicated they are involved in biological processes and pathways modulated by HQ. Taken together, these observations, which were consistent across two HQ dosages tested, suggest RSG co-treatment moderated cellular transcriptome changes elicited by HQ.

The expression of HO-1 protein is highly up-regulated by a variety of stress sources including oxidative stress, substrate heme, ultraviolet light, hyperthermia, heavy metals, peroxides, endotoxins and cytokines. HO-1 induction is recognized as a pivotal cellular adaptive and protective response against oxidative stress toxicity. HMOX-1 mRNA levels are significantly increased in undifferentiated and differentiated ARPE-19 cells treated with t-butyl hydroperoxide (tBH) and hydrogen peroxide, while HO-1 protein expression is up-regulated in ARPE-19 cells treated with cigarette smoke extract. ARPE-19 cells that overexpress HO-1 are more resistant to tBH-induced cell death and HO-1 activation has shown to play an important role to protect against retinal injury. In this study, we found that HMOX-1 gene and HO-1 protein expression were significantly up-regulated by HQ and further increased by RSG co-treatment. Additional up-regulation of HO-1 by RSG co-treatment suggests that RSG triggers the activation of antioxidant enzyme in response to oxidative stress. However, activation of HO-1 was only observed when cells were under HQ stress, as RSG alone did not induce observable cellular stress and HO-1 activation. Further studies are needed to investigate the precise upstream molecular regulator of HO-1 activation induced by HQ and RSG co-treatment.

In conclusion, RSG co-treatment protects RPE cells against HQ-induced injury, restores mitochondrial function, and modifies a variety of the biological processes induced by HQ. While some of these observations can be explained by regulation of integrin by RSG, the precise molecular mechanisms governing the promotion of cell survival by RSG remain to be fully elucidated. We continue to investigate the RSG-modulated biological pathways and believe this information will enhance our understanding of a potential role for RSG therapy to treat degenerative retinal diseases, such as dry AMD.

### Example 2

### Improving Myocardial Function/Treating Heart Failure

Normal heart function requires the cardiac myocytes to receive an adequate supply of energy in the form of adenosine triphosphate (ATP). However, cardiac myocytes store relatively small amounts of ATP. Therefore, the mitochondria of cardiac myocytes must continually synthesize ATP to maintain normal cardiac function. In at least some types of heart failure, the mitochondria of cardiac myocytes fail to produce enough ATP to meet myocardial energy demand. This results in a buildup of mitochondrial reactive oxygen species and oxidative stress followed by necrosis of cardiac myocytes, pathologic tissue remodeling and left ventricular dysfunction. Therefore, administration of effective amounts of peptide(s) as disclosed herein will lessen the mitochondrial ATP deficit and oxidative stress due to heart failure, thereby improving myocardial contractility recovery of cardiac function.

An initial study was conducted to assess the effects of Risuteganib administered intravenously at doses of 1.0 mg/kg and 5.0 mg/kg in a dog heart failure model. The subject animals underwent coronary microembolization to induce heart failure as described in Sabbah, H., et al.; Chronic Therapy with Elamipretide (MTP-131), a Novel Mitochondria-Targeting Peptide, Improves Left Ventricular and Mitochondrial Function in Dogs with Advanced Heart Failure; Circ Heart Fail. 2016 February; 9(2): e002206. doi:10.1161/CIRCHEARTFAILURE.115.002206.

Three (3) dogs were received 1.0 mg/kg Risuteganib every 2 weeks for 6 weeks. After an intervening period of approximately one week, the same three (3) dogs then received 5.0 mg/kg Risuteganib every 2 weeks for 6 additional weeks. Ventriculographic and hemodynamic measurements were made prior to the first intravenous dose of Risuteganib, after the third biweekly 1.0 mg/kg intravenous dose of Risuteganib and again after the third biweekly 5.0 mg/kg intravenous dose of Risuteganib. The following Table 1 shows the ventriculographic and hemodynamic data generated in this study:

TABLE 1

| | | | Risuteganib | | Risuteganib |
|---|---|---|---|---|---|
| | Control Dogs | | 1.0 mg/kg, IV | | 5.0 mg/kg, IV |
| | (Published Data) | | q 2 weeks for 6 weeks | | q 2 weeks for 6 weeks |
| | Pretreatment | 3 months | Pretreatment | 6 weeks post 3^{rd} 1.0 | 6 weeks post 3^{rd} 5.0 mg.kg |
| Parameters | (n = 7) | Post IPdaily | (n = 3) | mg/kg IV inj. | IV inj. |
| Left Ventricle | 31 | 29 | 32 | 37 | 41 |
| Ejection | | Δ = −2 | | Δ = +5 | Δ = +9 |
| Fraction % | | | | | |
| Stroke Volume | 19 | 21 | 21 | 23 | 27 |
| mL | | Δ = +2 | | Δ = +2 | Δ = +5 |
| Cardiac Output | 1.7 | 1.76 | 1.81 | 1.95 | 2.38 |
| (L/min) | | Δ = +0.06 | | Δ = +0.14 | Δ = +0.57 |
| L V End | 63 | 69 | 64 | 63 | 67 |
| Diastolic | | | | | |
| Volume (mL) | | | | | |
| L V End | 44 | 48 | 43 | 40 | 39 |
| Systolic | | | | | |
| Volume (mL) | | | | | |
| Heart Rate | 88 | 84 | 88 | 86 | 87 |
| (beats/min) | | | | | |
| Mean Aortic | 74 | 74 | 75 | 71 | 84 |
| Pressure | | | | | |
| (mmHg) | | | | | |
| L V Peak + | | | 1241 | 1267 | 1288 |
| dP/dt | | | | | |
| (mmHg/sec) | | | | | |
| L V Peak + | | | 1509 | 1325 | 1338 |
| dP/dt | | | | | |
| (mmHg/sec) | | | | | |
| L V End | 15 | 16 | 13 | 13 | 13 |
| Diastolic | | | | | |
| Pressure | | | | | |
| (mmHg) | | | | | |
| Systemic | 4223 | 3855 | 3391 | 2954 | 2924 |
| Vascular | | | | | |
| Resistance | | | | | |
| (dynes-sec-cm- | | | | | |
| 5) | | | | | |
| Mean | | | 17 | 16 | 16 |
| Pulmonary | | | | | |
| Artery | | | | | |
| Pressure | | | | | |
| (mmHg) | | | | | |
| Mean | | | 9.3 | 9.0 | 9.0 |
| Pulmonary | | | | | |
| Artery Wedge | | | | | |
| Pressure | | | | | |
| (mmHg) | | | | | |

These preliminary data confirm that cardiac function improved in the heat failure dogs treated intravenously with 1.0 mg/kg and 5.0 mg/kg Risuteganib.

Based on the data summarized in this patent application, the peptide treatments disclosed herein, including RSG, may be administered to human or non-human animals to improve or prevent impairment of mitochondrial bioenergetics. This may be of benefit in the treatment of a number of disorders which are related to or characterized by diminished mitochondrial function or mitochondrial impairment. Disorders that may be treated using the herein disclosed peptides such as RSG include not only age related macular degeneration, retinal disorders and heart failure, as described above, but also a wide variety of other disorders, some non-limiting examples of which are described below:

### Treatment of Disorders Caused by Chemical, Hypoxic or Ischemic Insult

The peptide treatments disclosed herein may be administered before, during or after a chemical, hypoxic or ischemic events, thereby lessening the resultant cell damage, cell death, apoptosis, reperfusion injury, secondary injury or other damage resulting from such events. Such may include preservation of neurons of the central or peripheral nervous systems following such events. For example, hypoxic or ischemic injuries to the infant brain during or around the time of birth have been implicated in the manifestation of long-term neurological disorders and disabilities such as cerebral palsy, learning disability and behavioral disorders. Mitochondrial dysfunction is believed to contribute to the development of brain inflammation and apoptosis following perinatal hypoxic or ischemic insult. Leaw, B., et al.; Mitochondria, Bioenergetics and Excitotoxicity: New Therapeutic Targets in Perinatal Brain Injury; Front. Cell. Neurosci. 11:199 doi: 10.3389/fncel.2017.00199. Thus, peptide treatment as disclosed herein may serve to lessen the mitochondrial dysfunction that follows a chemotoxic, hypoxic or ischemic event thereby improving neuronal survival and function following such event.

### Treatment of Neurodegenarative Diseases

Neurodegenerative diseases including but not limited to including Alzheimer's disease (AD), Parkinson's disease (PD), Huntington's disease (HD) and amyotrophic lateral sclerosis (ALS) are characterized by the progressive degeneration of function and structure of the central nervous system (CNS). Impaired mitochondrial function is believed to play a significant role in the pathogenesis and/or progression of neurodegenerative diseases. Because of their high energy requirements, neurons of the CNS are especially vulnerable to injury and death due to mitochondrial dysfunction. Also, mitochondrial electron transport chain (ETC.) complexes have been associated with misfolding protein aggregation in at least some types of neurodegenerative diseases. See, Golpich, M, et al.: Mitochondrial Dysfunction and Biogenesis in Neurodegenerative diseases: Pathogenesis and Treatment; CNS Neuroscience & Therapeutics 23 (2017) 5-22. Thus, peptide treatment as disclosed herein may effectively treat and lessen the severity/progression of such neurodegerative diseases.

### Treatment of Peripheral Neuropathies

The peptide treatments disclosed herein may be used to treat various types of peripheral neuropathy and peripheral nerve pain, such as peripheral nerve damage resulting from certain cancer treatments (e.g., cisplatin administration) and diabetic neuropathy. The neurons and associated distal axons of the peripheral nervous system are highly dependent on energy. Conditions such as diabetes can interfere with glucose and lipid energy metabolism with resultant mitochondrial (Mt) dysfunction and impaired energy homeostasis in peripheral nerves and, over long term, degeneration and oxidative injury to neurons and axons.

One specific pathway believed to be involved in the etiology of diabetic peripheral neuropathy (DPN) is the energy sensing pathway known as the nicotinamide-adenine dinucleotide (NAD⁺)-dependent Sirtuin 1 (SIRT1)/peroxisome proliferator-activated receptor-γ coactivator a (PGC-1α)/Mt transcription factor A (TFAM or mtTFA) signaling pathway. Chandrasekaran, K., et al.; Role of mitochondria in diabetic peripheral neuropathy: Influencing the NAD⁺-dependent SIRT1-PGC-1α-TFAM pathway; Int Rev Neurobiol. 2019; 145: 177-209. doi:10.1016/bs.irn.2019.04.002.

Chemotherapy-induced peripheral neuropathy can be a painful side effect of cancer treatment. Mitochondrial dysfunction and oxidative stress have been identified as factors in the development of chemotherapy-induced peripheral neuropathy. Cheng, X., et al.; Chemotherapy-induced peripheral neurotoxicity and complementary and alternative medicines: Progress and perspective; Frontiers in Pharmacology, 23 Oct. 2015; https://doi.org/l0.3389/fphar.2015.00234.

Based on the data set forth above, the peptide treatments described herein may be used to treat peripheral neuropathies and peripheral nerve pain.

### Treatment of Dermatologic Disorders

Primary mitochondrial disorders may sometimes cause skin manifestations (e.g., rashes, pigmentation abnormalities, acrocyanosis) and primary skin disorders may sometimes be linked to mitochondrial dysfunction. A number of skin disorders (e.g., pruritis, atopic dermatitis, psoriasis) may benefit from treatment, as described herein, to improve mitochondrial function. Mitochondrial dysfunction has been characterized as the rule rather than the exception in skin diseases. Feichtinger, R. G., et al.; Mitochondrial dysfunction: a neglected component of skin diseases; Experimental Dermatology Vol. 23, Issue 9, September 2014 (607-614); https://doi.org/10.1111/exd.12484

Based on the data set forth above, the peptide treatments described herein may be used to treat a variety of skin disorders including, for example, pruritis, atopic dermatitis and psoriasis.

### Effective Peptides Other Than Risuteganib

The effects and mechanisms of action referred to in this provisional patent application are not necessarily limited to Risuteganib. Other peptides, including those described in the above-incorporated patents and applications, are included in this disclosure. For example, peptides described in United States Patent Application Publication No. 2019/0062371 may reasonably be expected to also exhibit the herein described effects and/or mechanisms of action. Specific examples of other peptides believed to exhibit some or all of these effects or mechanisms include, but are not necessarily limited to, comprise peptides that consist of or comprise an amino acid sequence having the formula:

Y—X—Z
wherein: Y═R, H, K, Cys(acid), G or D;
X=G, A, Cys(acid), R, G, D or E; and
Z=Cys(acid), G, C, R, D, N or E.

Such peptides may comprise or consist of the amino acid sequences; R-G-Cys(Acid), R—R-Cys, R-CysAcid)-G, Cys(Acid)-R-G, Cys(Acid)-G-R, R-G-D, R-G-Cys(Acid). H-G-Cys(Acid), R-G-N, D-G-R, R-D-G, R-A-E, K-G-D, R-G-Cys(Acid)-G-G-G-D-G, Cyclo-{R-G-Cys(acid)-F—N-Me-V}, R-A-Cys (Acid), R-G-C, K-G-D, Cys(acid)-R-G, Cys(Acid)-G-R, Cyclo-{R-G-D-D-F—NMe-V}, H-G-Cys(acid) and salts thereof. Possible salts include but are not limited to acetate, trifluoroacetate (TFA) and hydrochloride salts.

Also, other peptides believed to exhibit some or all of these effects or mechanisms include, but are not necessarily limited to, those that consist of or comprise comprises linear or cyclic forms of Glycinyl-Arginyl-Glycinyl-Cysteic acid-Threonyl-Proline-COOH or which have the formula:

XI—R-G-Cysteic Acid-X
wherein: X and XI are selected from: Phe-Val-Ala, -Phe-Leu-Ala, -Phe-Val-Gly, -Phe-Leu-Gly, -Phe-Pro-Gly, -Phe-Pro-Ala, -Phe-Val; or from Arg, Gly, Cysteic acid, Phe, Val, Ala, Leu, Pro, Thr and salts, and any combinations of any D-isomers and L-isomers thereof.

It is to be appreciated that, although the invention has been described hereabove with reference to certain examples or embodiments of the invention, various additions, deletions, alterations and modifications may be made to those described examples and embodiments without departing from the intended spirit and scope of the invention. For example, any elements, steps, members, components, compositions, reactants, parts or portions of one embodiment or example may be incorporated into or used with another embodiment or example, unless otherwise specified or unless doing so would render that embodiment or example unsuitable for its intended use. Also, where the steps of a method or process have been described or listed in a particular order, the order of such steps may be changed unless otherwise specified or unless doing so would render the method or process unsuitable for its intended purpose. Additionally, the elements, steps, members, components, compositions, reactants, parts or portions of any invention or example described herein may optionally exist or be utilized in the absence or substantial absence of any other element, step, member, component, composition, reactant, part or portion unless otherwise noted. All reasonable additions, deletions, modifications and alterations are to be considered equivalents of the described examples and embodiments and are to be included within the scope of the following claims.

1. A method for improving mitochondrial bioenergetics in a human or animal subject in need thereof, said method comprising the step of administering to the subject a therapeutically effective amount of a peptide which causes an improvement in mitochondrial bioenergetics.

2. A method according to claim 1 wherein the subject suffers from a disorder which causes or is caused by impairment of mitochondrial bioenergetics and wherein the administration of the peptide reverses or prevents at least some of the impairment of mitochondrial bioenergetics causing or caused by said disorder.

3. A method according to claim 2 wherein the disorder comprises or results from a chemotoxic, hypoxic or ischemic insult.

4. A method according to claim 2 wherein the disorder comprises or results from metabolic stress.

5. A method according to claim 2 wherein the disorder comprises heart failure, kidney failure and kidney disease.

6. A method according to claim 2 wherein the disorder comprises a neurodegenerative disease.

7. A method according to claim 6 wherein the neurodegerative disease is selected from: Alzheimer's disease (AD), Parkinson's disease (PD), Huntington's disease (HD), amyotrophic lateral sclerosis (ALS) and other disorders which cause progressive degeneration of function and/or structure of neurons of the central nervous system (CNS).

8. A method according to claim 2 wherein the disorder comprises a dermatologic disorder.

9. A method according to claim 8 wherein the dermatologic causes or is accompanied by a rash, pigmentation abnormality or acrocyanosis

10. A method according to claim 8 wherein the dermatologic disorder comprises: pruritis, atopic dermatitis or psoriasis.

11. A method according to claim 2 wherein the disorder comprises a peripheral neuropathy or peripheral nerve pain.

12. A method according to claim 11 wherein the peripheral neuropathy or peripheral nerve pain is causes or is caused by: mitochondrial dysfunction, diabetes, abnormal glucose metabolism, oxidative stress or chemotherapy.

13. A method according to claim 2 wherein the disorder comprises heart failure or reduced cardiac output.

14. A method according to claim 1 wherein the peptide comprises Risuteganib.

15. A method according to claim 1 wherein the peptide comprises a linear or cyclic form of Glycinyl-Arginyl-Glycinyl-Cysteic (acid)-Threonyl-Proline-COOH

16. A method according to claim 1 wherein the peptide has the formula:
X1-Arg-Gly-Cysteic Acid-X
wherein X and X1 are selected from: Phe-Val-Ala, -Phe-Leu-Ala, -Phe-Val-Gly, -Phe-Leu-Gly, -Phe-Pro-Gly, -Phe-Pro-Ala, -Phe-Val; or from Arg, Gly, Cysteic acid, Phe, Val, Ala, Leu, Pro, Thr and salts, and any combinations of any D-isomers and L-isomers thereof.

17. A method according to claim 1 wherein the peptide has the general formula:
Y—X—Z
wherein: Y═R, H, K, Cys(acid), G or D;
X=G, A, Cys(acid), R, G, D or E; and
Z=Cys(acid), G, C, R, D, N or E.

18. A method according to claim 1 wherein the peptide comprises or consists of an amino acid sequence selected from: R-G-Cys(Acid), R—R-Cys, R-CysAcid)-G, Cys(Acid)-R-G, Cys(Acid)-G-R, R-G-D, R-G-Cys(Acid). H-G-Cys(Acid), R-G-N, D-G-R, R-D-G, R-A-E, K-G-D, R-G-Cys(Acid)-G-G-G-D-G, Cyclo-{R-G-Cys(acid)-F—N-Me-V}, R-A-Cys (Acid), R-G-C, K-G-D, Cys(acid)-R-G, Cys(Acid)-G-R, Cyclo-{R-G-D-D-F—NMe-V}, H-G-Cys(acid) and salts thereof.

19. (canceled)

20. A method according to claim 1 wherein the peptide comprises Risuteganib and wherein the subject suffers from a disorder selected from: a chemotoxic, hypoxic or ischemic insult, metabolic stress, heart failure, chronic heart failure with reduced ejection fraction, chronic heart failure with preserved ejection fraction, Barth syndrome, kidney disease, kidney failure due to percutaneous renal angiography for renal artery stenosis, impaired skeletal muscle function, primary muscle mitochondrial myopathy or neuropathy, ischemia-reperfusion injury, protozoal infections, peripheral neuropathy, dermatologic disorders, neuronerative disease, Alzheimer's disease (AD), Parkinson's disease (PD), Huntington's disease (HD), amyotrophic lateral sclerosis (ALS), another disorder which causes progressive degeneration of function and/or structure of neurons of the central nervous system (CNS), a dermatologic disorder, a rash, pigmentation abnormality or acrocyanosis, pruritis, atopic dermatitis, psoriasis, a peripheral neuropathy, peripheral nerve pain, or nerve pain that causes, contributes to, or is caused by mitochondrial dysfunction, diabetes, abnormal glucose metabolism, oxidative stress or chemotherapy, heart failure or reduced cardiac output.
